(19) United States
(12) Patent Application Publication
(10) Pub. No.: US 2010/0112719 A1
(43) Pub. Date: May 6, 2010

(54) ELECTRONIC SIGNAL AMPLIFICATION IN FIELD EFFECT DEVICE BASED CHEMICAL SENSORS

(76) Inventors: Amihood Doron, Ahuzat Barak (IL); Ilan Levy, Harif (IL)

Correspondence Address: COOL PATENT, P.C.;c/o CPA Global, P.O. BOX 52050, MINNEAPOLIS, MN, 55402, US

(21) Appl. No.: 12/266,371

(22) Filed: Nov. 6, 2008

(51) Int. Cl. G01N 33/566; G01N 27/00; B01J 19/00; C12M 1/34; G01N 27/26; G01N 33/543

(52) U.S. Cl. 436/501; 422/82.01; 422/68.1; 435/287.1; 204/400; 436/518

(57) ABSTRACT

Briefly, disclosed is a method and apparatus for detecting an analyte wherein an enhanced charge marker may enhance steric, electrostatic, optic and/or mechanical changes associated with a recognition event between an analyte and a probe.

### BACKGROUND

### Technical Field

The disclosure relates to chemical sensors, more particularly the disclosure relates to solid state sensors capable of chemical sensing by detection of electrostatic changes associated with a recognition event.

### BRIEF DESCRIPTION OF THE DRAWINGS

FIG. 1 is a diagram illustrating a particular embodiment of an analyte coupled to an enhanced charge marker.

FIG. 2 is a diagram illustrating a particular embodiment of a secondary probe coupled to an enhanced charge marker.

FIG. 3 is a diagram illustrating a particular embodiment of a chemical sensor capable of detecting an analyte coupled to an enhanced charge marker.

FIG. 4 is a diagram illustrating a particular embodiment of a chemical sensor capable of detecting an analyte via a secondary probe coupled to an enhanced charge marker.

FIG. 5 is a diagram illustrating a particular embodiment of a system comprising a sensor to detect an analyte.

FIG. 6 is a block diagram illustrating a particular embodiment of a process for detecting the presence of an analyte in a sample.

### DETAILED DESCRIPTION

In the following detailed description, numerous specific details are set forth in order to provide a thorough understanding of claimed subject matter related to chemical sensors comprising field effect devices capable of detection of electrostatic changes associated with chemical recognition events. However, it will be understood by those skilled in the art that claimed subject matter may be practiced without these specific details. In other instances, well-known methods, procedures, and components have not been described in detail so as not to obscure claimed subject matter.

Throughout the following disclosure the term ‘chemical sensor’ is used and is intended to refer to a device capable of detection of an analyte that combines a detecting component or ‘probe’ with an electronic and/or mechanical detector element. The terms ‘biomolecule’ and ‘biomolecular’ are used throughout the following disclosure and are intended to refer to one or more molecules that may be biologically active and may be naturally occurring in living organisms or may be synthesized by a variety of non-naturally occurring methods. The term ‘analyte’ is used throughout the following disclosure and is intended to refer to any chemical, biochemical and/or biomolecular substance that is undergoing analysis.

The term ‘molecular recognition event’ or ‘recognition event’ is used throughout the following disclosure and is intended to refer to an interaction between a probe or capture molecule and an analyte giving rise to a specific and/or selective recognition of an analyte. Molecular recognition or specific recognition refers to the specific interaction between two or more molecules typically through non-covalent bonding interactions such as hydrogen bonding, metal coordination, hydrophobic forces, van der Waals forces, π-π interactions, and or electrostatic effects. Two molecules that are able to undergo a molecular recognition event are referred to as having molecular complementarity. Molecular complementarity is sometimes thought of as being similar to the way a key fits into a lock in that a key has a specific shape that is designed for and capable of interacting with a specific lock. Examples of molecular recognition events include receptor-ligand, antigen-antibody and sugar-lectin.

The terms target, target molecule, or analyte refer to a molecule of interest that is to be detected. The terms probe, probe molecule, or capture molecule refer to a molecule that selectively recognizes or binds to a target molecule or undergoes a chemical reaction with a target molecule. The probe or probe molecule generally, but not necessarily, has a known molecular structure or sequence. Probe molecules are molecules capable of undergoing binding or molecular recognition events with target molecules. Probes may be naturally-occurring or synthetic molecules. Probes can be employed in their unaltered state or as aggregates with other species. Examples of probes which can be used in conjunction with the disclosed method and device include, but are not limited to, antibodies, peptides, proteins, enzymes, receptors, targets, pharmaceutical drugs, cofactors, lectins, sugars, polysaccharides, cells, cellular membranes, and organelles. The probe molecule or the target molecule can be a ligand or a receptor. A ligand is a molecule that typically binds to another molecule, usually referred to as a receptor, the level of specificity can vary. Usually, the term ligand is given to the smaller of the two molecules in the ligand-receptor pair, but it is not necessary for this to be the case. A receptor can be considered to be a molecule that has an affinity for a particular ligand. Typically, in a cell, a receptor is a protein molecule to which a mobile signaling molecule can specifically bind. Cellular receptors include opiate receptors, neurotransmitter receptors, steroid receptors, intracrine peptide hormone receptors, and hormone receptors. Examples of ligands include, but are not limited to, agonists and antagonists for cell membrane receptors, toxins and venoms, viral epitopes, hormones, hormone receptors, peptides (such as neurotransmitters), cofactors, pharmaceutical drugs, lectins, sugars, and oligosaccharides.

Throughout the following disclosure particular embodiments of solid-state chemical sensors are disclosed. Biomolecular sensors for detecting analytes comprising various chemical and biomolecular compounds are discussed. Particular embodiments of the device and method disclosed herein may be useful for detecting many varieties of organic and inorganic chemicals, biochemicals and/or biomolecules using a variety organic and inorganic chemicals and compounds such as probes or capture molecules and claimed subject matter is not limited in this regard. Such analytes and probes may be naturally occurring and/or synthetic, organic and/or inorganic chemicals, biochemicals and/or biomolecules and claimed subject matter is not limited in this regard.

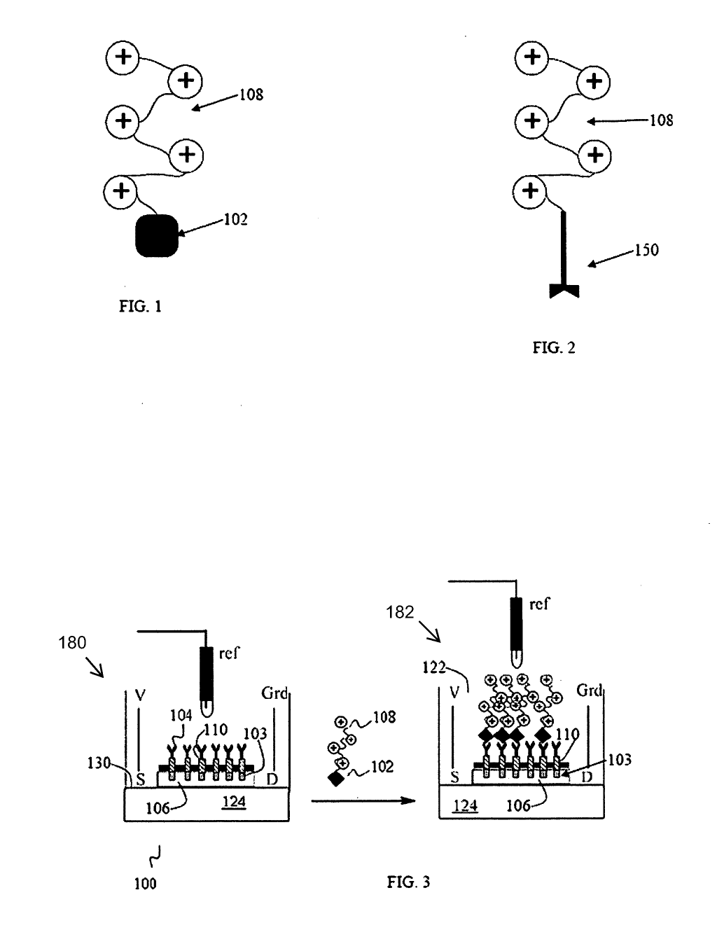

FIG. 1 illustrates a particular embodiment of an analyte 102 coupled to enhanced charge marker (ECM) 108. In a particular embodiment, ECM 108 is a molecule carrying a net positive and/or negative charge. ECM 108 may be an electrostatic marker configured to be coupled to an analyte 102. In another embodiment, ECM 108 may be coupled to a probe (see FIG. 2) and claimed subject matter is not limited in this regard.

In a particular embodiment, ECM 108 may be used to mark a probe and/or analyte for chemical assay using a chemical sensor wherein detection of an analyte depends on detecting a physical change associated with a recognition event. During such a chemical assay, ECM 108 is operable to amplify steric, electrostatic and/or mechanical changes during the recognition event between the probe and analyte thus increasing the sensitivity and/or selectivity of the assay.

A chemical sensor may detect analyte 102 by detecting various physical changes associated with molecular recognition events between a probe and analyte. According to a particular embodiment, ECM 108 may be engineered or synthesized to carry a net charge sufficient to enhance the physical changes associated with molecular recognition events during detection. Such physical changes or effects may include steric, electrostatic, conformational, charge and/or conductivity affects. In an embodiment, ECM 108 comprises a net charge greater than 2 or less than −2.

In a particular embodiment an ECM 108 may be selected based at least in part on net charge of a target analyte and/or probe to which the ECM is to be coupled. ECM 108 may be a substance different from the target analyte and/or probe. In the following examples the target analyte and/or probe may be any of a variety of species that have a net charge in the specified range and claimed subject matter is not limited in this regard.

For example, in a particular embodiment in a working solution having a pH in the range of 3-10 where the analyte or probe has a net charge in the range of about −20 to 20, an ECM 108 comprising an effective charge in the range of about −10 to −2 or about 2 to 10 may be selected to be coupled to the analyte and/or probe. Effective charge may be a net charge of ECM 108 after subtracting the net charge of the analyte or probe. In this embodiment, ECM 108 may improve the sensitivity of a chemical sensor by enhancing physical changes associated with molecular recognition events during detection of an analyte as described above. ECM 108 may comprise, for example, a polypeptide or other polymer comprising about 20 mers in one embodiment, but claimed subject matter is not limited in this regard.

In another particular embodiment in a working solution having a pH in the range of 3-10 where the analyte or probe has a net charge in the range of about −40 to 40, an ECM 108 comprising an effective charge in the range of about −15 to −2 or about 2 to 15 may be selected to be coupled to the analyte and/or probe. In this embodiment, ECM 108 may improve the sensitivity of a chemical sensor by enhancing physical changes associated with molecular recognition events during detection of an analyte as described above. ECM 108 may comprise, for example, a polypeptide or other polymer comprising about 30 mers in one embodiment, but claimed subject matter is not limited in this regard.

In another particular embodiment in a working solution having a pH in the range of 6-8 where the analyte or probe has a net charge in the range of about −3 to 3 (such as a protein), an ECM 108 comprising an effective charge in the range of −15 to −2 or about 2 to 15 may be selected to be coupled to the analyte and/or probe. In this embodiment, ECM 108 may improve the sensitivity of a chemical sensor by enhancing physical changes associated with molecular recognition events during detection of an analyte as described above. ECM 108 may comprise, for example, single-stranded DNA (ssDNA) or other polymer comprising about 15 mers in one embodiment, but claimed subject matter is not limited in this regard.

In another particular embodiment in a working solution having a pH in the range of 6-8 where the analyte or probe has a net charge in the range of about −10 to 10 (such as various metabolites), an ECM 108 comprising an effective charge in the range of about −25 to −2 or about 2 to 25 may be selected to be coupled to the analyte and/or probe. In this embodiment, ECM 108 may improve the sensitivity of a chemical sensor by enhancing physical changes associated with molecular recognition events during detection of an analyte as described above. ECM 108 may comprise, for example, ssDNA or other polymer comprising about 25 mers in one embodiment, but claimed subject matter is not limited in this regard.

ECM 108 may comprise phosphate, carboxylate, amine and/or sulfonate groups and claimed subject matter is not limited in this regard. In a particular embodiment, phosphate, carboxylate, and/or sulfonate groups may be negatively charged under biological conditions (ph 6.0-8.0) whereas amine groups may comprise positive charges under biological conditions.

In another particular embodiment, ECM 108 may comprise an artificial and/or native polymer backbone with functional groups. Such polymer backbone may comprise a variety of species including; negative DNA (deoxyribonucleic acid) oligomers, negative or positive peptide oligomers and/or synthetic polymers with positive or negative side groups. In a particular embodiment, such side groups may comprise sulfonate and/or carboxylated aliphatic and aromatic amines and/or heterocyclic-organometalic complexes. Additionally, in a particular embodiment, a ECM 108 may be chosen to comprise a higher or lower pH than the pH of sample medium 122 (working solution).

In another embodiment, ECM 108 may comprise a number of charged groups where the number of charged groups on ECM 108 is selected to be inversely proportional to the concentration of analyte 102 in sample medium 122. For example, if analyte 102 concentration is low in sample medium 122, an ECM 108 may be selected comprising a greater number of charged groups to enhance sensitivity of a chemical sensor. In another example, if analyte concentration is high, ECM 108 may be selected comprising a lower number of charge groups to minimize unintended interactions between charged groups.

According to a particular embodiment, ECM 108 may be coupled to analyte 102 by a variety of methods such as, for instance, carbodiimide coupling of sDNA, peptide nucleic acid (PNA), and/or a peptide unit to analyte 102 and/or secondary probe 150. In another particular embodiment, short peptide or PNA sequences may be coupled to ECM 108 comprising other charged species as described above. Such short peptide or PNA sequences may be engineered such that they may bind to the analyte by specific interaction on specific locations. However, these are merely examples of a variety of enhanced charge markers and claimed subject matter is not limited in this regard.

According to a particular embodiment, marking analyte 102 with ECM 108 may enable enhanced detection of analyte 102 by chemical sensors capable of recognizing analyte 102 wherein the chemical sensor detects the presence of analyte 102 by sensing steric and/or electrostatic changes brought about during a recognition event. Such sensors may comprise a variety of devices and/or materials capable of detecting steric, electrostatic, conformational, charge and/or conductivity changes by enhancing an electrostatic charge at an interface upon analyte 102 interaction with probe 150. Such physical changes may activate a transducing mechanism of a sensor. Such chemical sensors may be field effect transistors, piezo-electric materials, crystal material, ion-sensitive field effect transistors (ISFET), electrolyte-insulator-semiconductor (EIS), amperometric or potentiometer electrode sensor, capacitance sensor and/or reflectance and refractive sensors (for example, surface plasmon resonance (SPR), Elipsometery, etc) and claimed subject matter is not limited in this regard.

FIG. 2 illustrates a particular embodiment of secondary probe 150 coupled to charged ECM 108. In a particular embodiment, secondary probe 150 coupled to ECM 108 may enable enhanced detection of an analyte (not shown). In a particular embodiment, ECM 108 may be a substance different from secondary probe 150. In a particular embodiment, an enhanced charge of ECM 108 may enable an increased sensitivity in chemical sensors capable of detecting the presence of an analyte by detecting physical changes such as steric, electrostatic, conformational, charge and/or conductivity affects associated with a recognition event between an analyte immobilized on one or more probes of the chemical sensor and secondary probe 150. According to a particular embodiment, such a chemical sensor may be capable of enhance detection because steric, electrostatic, conformational, charge and/or conductivity affects corresponding to a recognition event between secondary probe 150 and an immobilized analyte may be exaggerated by the presence of an enhanced charge on ECM 108.

FIG. 3 illustrates an embodiment of sensor 100 during detection of analyte 102 marked with a poly-charged ECM 108. Block 180 on the left depicts sensor 100 before exposure to a sample 122 containing analyte 102. Block 182 on the right depicts sensor 100 after exposure to sample 122 where probes 104 and analyte 102 have undergone a recognition event and analyte 102 is coupled to probes 104.

In a particular embodiment, sensor 100 may be a chemical sensor capable of detecting a variety of chemical, biochemical and biomolecular species and claimed subject matter is not limited in this regard. In a particular embodiment, sensor 100 may comprise one or more embedded field effect devices (FED) 103 disposed in substrate 106. Sensor 100 may comprise a single FED 103 or may comprise an array of FEDs 103 (as shown in FIG. 3). Such FEDs 103 may comprise field effect transistors, piezo-electric materials, crystal material, ion-sensitive field effect transistors (ISFET), and/or electrolyte-insulator-semiconductor (EIS) devices and claimed subject matter is not limited in this regard. FED 103 may be sterically and/or electrostatically sensitive and may be coupled to a capture molecule such as a probe 104. Sensor 100 may be fabricated in a variety of dimensions, such as, microscale or nanoscale fabrication and claimed subject matter is not limited in this regard.

In a particular embodiment, probe 104 may be directly in contact with the ambient, such as, sample 122. In a particular embodiment, probe 104 may be coupled to member 110 extending from substrate 106. Sensor 100 may be exposed to sample 122 containing analyte 102. Such a sample may be in solid, liquid and/or gas phase and may comprise a variety of species from which an analyte 102 may be differentiated. Sensitivity and specificity of sensor 100 may depend on a number of variables such as, for instance, the type of sterically and/or electrostatically sensitive device or material used and the specificity of probe 104 and claimed subject matter is not limited in this regard. In a particular embodiment, ECM 108 may enable enhanced detection of an analyte 102 with respect to the results that may be achieved without coupling analyte 102 to ECM 108.

In a particular embodiment, during a recognition event, probe 104 may be coupled to an outside surface of member 110 and may be capable of forming a bond to analyte 102 and thereby inducing electrostatic effects and mechanical stress on member 110 due to steric and/or electrochemical effects of bonding. In another particular embodiment, charge density rearrangement of analyte 102 may occur during such a recognition event. Such charge density rearrangement may change the net charge of analyte 102 and enable a surface potential on member 110. Such a change in the surface potential in member 110 may modulate channel conductivity in FED 103 by changing a voltage on a gate (not shown) of FED 103. In another particular embodiment, member 110 may be coupled to a variety of probes that may be capable of bonding to different analytes. Thus, sensor 100 as disclosed herein may be capable of detecting and/or recognizing one or more analytes to enable detection of different analytes in the same sample. However, these are merely examples of probe configurations for a biosensor and claimed subject matter is not so limited.

In a particular embodiment, probe 104 may be immobilized on one or more members 110 extending from substrate 106. Such members 110 may comprise a variety of structures such as cantilevers, blades, cylinders, flexible gate electrodes (FGE) and/or nanotubes and claimed subject matter is not limited in this regard. According to a particular embodiment, member 110 may be coupled to FED 103 and may be operable to translate steric, electrostatic, conformational, charge and/or conductivity changes related to a recognition event into a signal in FED 103. According to a particular embodiment, member 110 may comprise an FGE where such an electrode may comprise a selectively permeable or reactive coating, such as, for instance, a lipid bilayer, hydrogel, polyvinyl acetate (PVA) and polyethylene glycol (PEG) based functional polymers and/or polyelectrolyte and claimed subject matter is not limited in this regard. According to a particular embodiment, an inside surface 130 of sensor 100 may be coated with various selectively permeable and/or reactive coatings and claimed subject matter is not limited in this regard.

In a particular embodiment, probe 104 may comprise a variety of materials and/or compounds that if exposed to sample 122 may be capable of recognizing and/or detecting the presence of analyte 102 in sample 122 to a greater extent than other substances that may be found in sample 122. Such recognition and/or detection may comprise probe 104 bonding, binding and/or coupling to analyte 102 via covalent and/or non-covalent or other forces. In a particular embodiment, recognition and/or detection may comprise probe 104 exhibiting steric and/or electrostatic behavioral changes associated with the presence of analyte 102 in a sample. Such a recognition event may trigger conformational and/or electrostatic changes in probe 104 that may be translated to FED 103 and/or a FED 103 array. In a particular embodiment, detection may be measured by a signal induced by FED 103 in response to recognition of analyte 102.

In a particular embodiment, probe 104 may comprise a variety of biomolecular species, such as: antibodies, antibody fragments, single-chain antibodies, genetically engineered antibodies, artificial antibodies (for example, affibodies or phages caring binding peptides), peptide nucleic acids, proteins, peptides, binding proteins, receptor proteins, transport proteins, lectins, substrates, inhibitors, activators, ligands, hormones, neurotranamitters, growth factors, cytokines, carbohydrates, aptamers, lipids, lipid bilayers and/or charged polymers and claimed subject matter is not limited in this regard.

In one example embodiment, an ECM 108 comprises a sodium poly(aspartate) molecule. An ECM 108 comprising a sodium poly(aspartate) molecule may be formed, for example, by reacting a molecule comprising a backbone having repeating succinimide units with sodium hydroxide to form a carboxylated functional group on every monomer unit. ECM 108 comprising poly(aspartate) may be a linear molecule having the formula —[CH(CH₂CO₂Na)CONH]ₙ— where n comprises a value from about 20 to about 50, or about 50 to about 500, or about 500 to about 5000, or combinations thereof. In another embodiment, n comprises a value from about 10 to about 100. Other useful substituents may include amine and/or sulfonate groups. For example, ECM 108 may comprise polystyrene sulfonic acid, poly(2-acrylamido-2-methyl-1-propanesulfonic acid), poly(allylamine hydrochloride), or poly(acrylamido-N-propyltrimethylammonium chloride), however, claimed subject matter is not so limited.

An ECM 108 such as a sodium poly(aspartate) molecule may be attached to a probe 104 such as, for example, immunoglobulin G (IgG) comprising anti-PSA antibody (Prostate Specific Antigen). Such ECM 108 may be coupled with the antibody using, for example, a carbodiimide coupling reagent such as 1-Ethyl-3-(3-dimethylaminopropyl)-carbodiimide (EDC) to form an amide bond between the ECM 108 and the probe 104. A resulting theoretical net charge on such ECM-antibody complex may comprise one negative charge per unit monomer at pH higher than pKa values of about 4.5. Part of such theoretical net charge may be screened to an extent by counter ions and polarity of water molecules, for example, however, significant charge may remain unscreened to affect the device 103. Subject matter is not necessarily limited in this regard.

In a particular embodiment, analytes 102 may comprise a variety of biomolecular species, such as: amino acid, peptide, polypeptide, protein, glycoprotein, lipoprotein, antibody, sugar, carbohydrate, oligosaccharide, polysaccharide, fatty acid, lipid, hormone, metabolite, growth factor, cytokine, chemokine, receptor, neurotransmitter, antigen, allergen, antibody, substrate, metabolite, cofactor, inhibitor, drug, pharmaceutical, nutrient, biohazardous agent, infectious agent, prion, vitamin, heterocyclic aromatic compound, carcinogen, mutagen, waste product, virus, bacterium, Salmonella, Streptococcus, Legionella, E. coli, Giardia, Cryptosporidium, Rickettsia, spore, mold, yeast, algae, amoebae, dinoflagellate, unicellular organism, pathogen, prion and/or a cell and claimed subject matter is not limited in this regard.

In a particular embodiment, substrate 106 may be comprised of a variety of materials, such as, for instance: silicon, silicon-oxide, gallium arsenide, silicon germanium, silicon carbide, gallium phosphide and/or polysilicon and claimed subject matter is not so limited. According to a particular embodiment, substrate 106 may be disposed on a support structure 124. According to a particular embodiment, the assembly may be sealed with a coating 130 that may be substantially impermeable to a variety of substances in a variety of physical phases and claimed subject matter is not so limited.

According to a particular embodiment, coating 130 may comprise any of a variety of materials, such as, photo resists, polyimide, epoxy, metal nitride, metal oxide, or any other barrier materials know to those of skill in the art and claimed subject matter is not limited in this regard. Such coating 130 may enable sensor 100 to be immersed in a liquid or gas sample and to be used repeatedly while resisting wear and device failure. However, this is merely an example of a method of protecting a surface of substrate 106/support 124 assembly and claimed subject matter is not so limited.

In a particular embodiment, sensor 100 may be exposed to sample 122 by a variety of methods, such as, for instance, by titrating an aqueous sample 122 containing analyte 102 directly onto FED 103 array and claimed subject matter is not limited in this regard. In a particular embodiment, sensor 100 may be partially enclosed in a package (see FIG. 5). Such a package may be configured in a variety of ways to enclose all or a portion of sensor 100 and claimed subject matter is not limited in this regard.

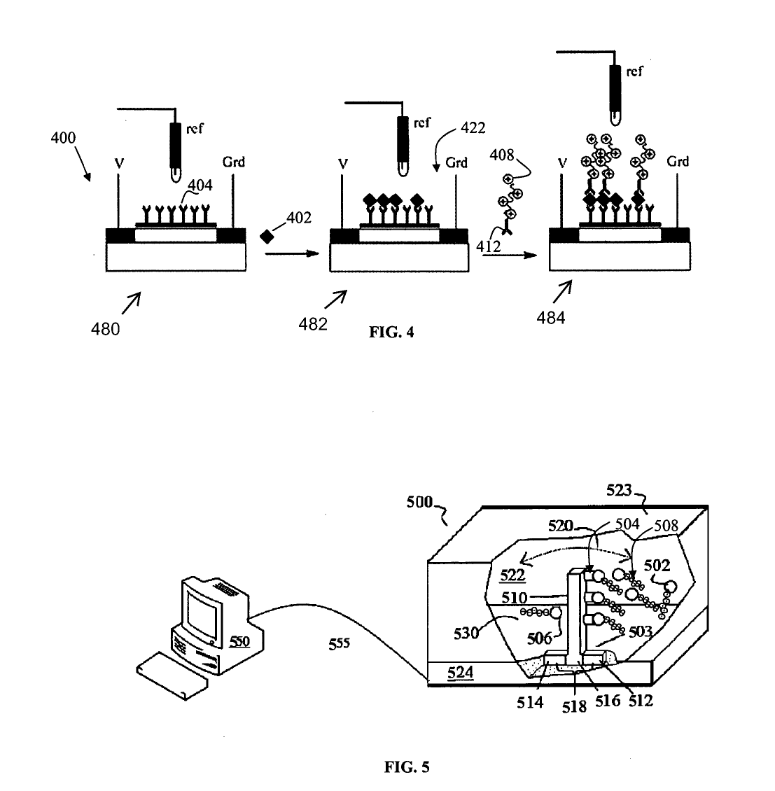

FIG. 4 illustrates an embodiment of sensor 400 during detection of analyte 402. In a particular embodiment, block 480 depicts sensor 400 prior to exposure to a sample 122 containing analyte 402. Block 482 depicts sensor 400 after exposure to sample 422 where probes 404 and analyte 402 have undergone a recognition event and analyte 402 is coupled to one or more probes 404. Block 484 depicts sensor 400 after sample 422 has been rinsed away and sensor 422 is exposed to a working solution comprising a secondary probe 412 coupled to an enhanced charge marker (ECM) 408. In this particular embodiment of a chemical assay using a secondary probe 412 an analyte 402 may be detected by sensor 400 if a recognition event between a secondary probe 412 and an immobilized analyte 402 occurs. Such an assay may have an increased specificity and/or selectivity due to use of two probes capable of undergoing recognition events in the presence of analyte 402. According to a particular embodiment, such an assay may also have an increased sensitivity due to an enhanced charge marker 408 marking the secondary probe 412. As described above, such an enhanced charge may increase steric and electrostatic effects brought on by a recognition event between a secondary probe and an analyte.

In a particular embodiment, secondary probe 412 may comprise monoclonal antibodies such as monoclonal antibodies raised against Prostate Specific Antigen (anti-PSA). In a particular embodiment, anti-PSA may be used to detect analyte 402 where analyte 402 is PSA. Such a secondary probe 412 may be coupled to ECM 408 by a variety of methods. For instance, ECM 108 may comprise peptide nucleic acid (PNA). In a particular embodiment, a PNA ECM 108 may be coupled to secondary probe 412 by coupling carbodiimide to the PNA. According to a particular embodiment, ECM 108 may have a variety of lengths. In one embodiment, ECM 108 comprises between about 5 mers to about 50 mers. Other lengths may be used in other embodiments and claimed subject matter is not limited in this regard. However, this is merely an example of marking a secondary probe with a particular electrostatic marker and claimed subject matter is not so limited.

FIG. 5 illustrates a particular embodiment of a sensor 500 for detecting analyte 502 wherein analyte 502 is electrostatically labeled with ECM 508. In a particular embodiment, ECM 508 may be a native or synthetic polymer comprising functional groups having a pKa lower (negative) or higher (positive) than the pH of a sample 522. Sensor 500 may be immersed in sample 522 within package 523. In a particular embodiment, sensor 500 may comprise FET 503 embedded in substrate 524. An outside surface of the substrate 524/FET 503 assembly may be sealed with impermeable coating 530. However, this is merely an example of a method of protecting a surface of the substrate 524/FET 503 assembly and claimed subject matter is not so limited.

In a particular embodiment, member 510 may be an extended gate electrode (FGE), may function as the FET 503 gate electrode and may be coupled to and extend from gate 516. In a particular embodiment, member 510 may be directly in contact with the ambient, such as, sample 522. Member 510 may have a substantially rectangular shape and may comprise or be coupled to an analyte sensitive material such as probe 504. According to a particular embodiment, probes 504 may be located on a single side of member 510 to enable mechanical stress to flex member 510 along arc 520. However, this is merely an example of a shape of a member 510 and a particular placement of probes 504 and claimed subject matter is not so limited.

Upon detection of analyte 502, member 510 may exert both mechanical stress on FET 503 and induce an electrostatic charge in gate 516. A specific recognition event between probes 504 and analyte 502 marked with ECM 508 may deflect FGE 510, along an arc 520 and may induce strain on FET 503 which may transform into conductivity effects in channel 518 of FET 503.

Sensor 500 may be immersed in sample 522 contained in package 523. Package 523 may be configured in a variety of ways and claimed subject matter is not limited in this regard. In a particular embodiment, sensor 500 may communicate detection of analyte 502 to a processing unit 550, such as a computer CPU and/or mobile unit processor and claimed subject matter is not limited in this regard. Communication may be via communication route 555 by any of a variety of communication techniques, such as for instance via wireline and/or wireless communication and claimed subject matter is not limited in this regard.

In another particular embodiment, sensor 500 may comprise a sensitive hydrogel (not shown). Such a hydrogel may be sensitive to a variety of stimuli and substances. Upon recognition of a substance or stimulus to which a hydrogel is sensitive, the volume of the hydrogel may change. According to a particular embodiment, member 510 may be in contact with a hydrogel and may undergo a change in volume upon sensing a probe 504 coupled to ECM 508. Such ECM 508 may amplify a volume change due to enhanced steric and/or electrostatic effects of charged marker ECM 508 on a hydrogel. In a particular embodiment, a hydrogel may deflect member 510, along an arc 520 and may induce strain on FET 503 which may transform into conductivity effects in channel 518. In a particular embodiment, such a hydrogel may be immobilized on a surface of member 510 and/or member 510 may be immersed in a sensitive hydrogel within an enclosed package. According to a particular embodiment, a sensitive hydrogel may comprise one or more biomolecular probes sensitive to one or more analytes. However, these are merely examples of a sensor 500 comprising a hydrogel and claimed subject matter is not limited in this regard.

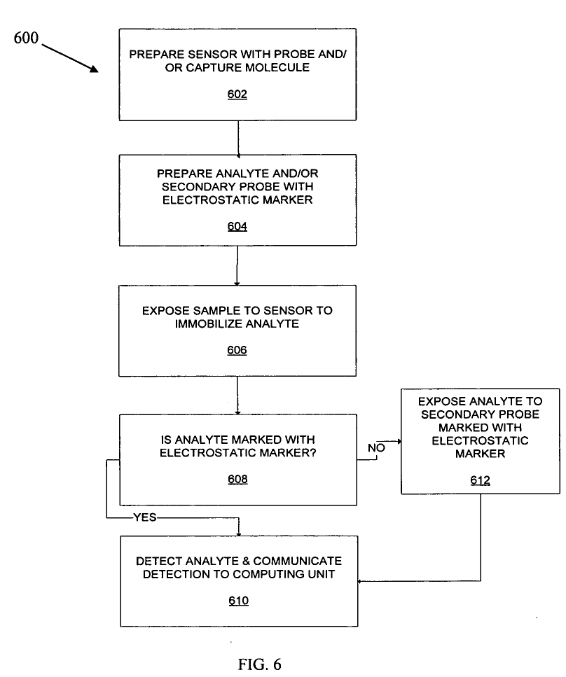

FIG. 6 is a block diagram illustrating a process 600 for detecting an analyte. At block 602, a sensor may be prepared by immobilizing a probe or capture molecule on a sensor surface and/or members extending from a sensor surface. Such probes or capture molecules may be capable of undergoing a recognition event with an analyte. According to a particular embodiment, process 600 may proceed to block 604 where a sample matrix (containing the analyte) and/or secondary probe may be prepared by marking with an electrostatic marker as described above. Process 600 need not proceed according in the order provide in FIG. 6. In a particular embodiment, analytes and secondary probes may be prepared at any time and claimed subject matter is not limited in this regard.

Process 600 may proceed to block 606 where a sample containing one or more analytes of interest may be exposed to the sensor. In a particular embodiment, exposing a sample to the sensor may immobilize an analyte on a surface of the sensor. Process 600 may proceed to block 608 where if the analyte is marked with an ECM process 600 may proceed to block 610 and if the analyte is not marked then process 600 may proceed to block 612.

At block 610 of process 600, an analyte may be detected by a sensor upon occurrence of a specific recognition event. During such a recognition event electrostatic and/or steric changes associated with the recognition event may translate to detecting devices of the sensor. When an analyte is detected, such detection may be communicated to a computing unit.

At block 612 of process 600 an analyte may be exposed to a secondary probe marked with an electrostatic marker. Process 600 may proceed to block 610 where an analyte may be detected by a sensor upon occurrence of a specific recognition event between the secondary probe and the analyte. During such a recognition event, electrostatic and/or steric changes associated with the recognition event may translate to detecting devices of the sensor. When an analyte is detected, such detection may be communicated to a computing unit.

While certain features of claimed subject matter have been illustrated as described herein, many modifications, substitutions, changes and equivalents will now occur to those skilled in the art. It is, therefore, to be understood that the appended claims are intended to cover all such embodiments and changes as fall within the spirit of claimed subject matter.

1. A method, comprising:
marking a substance with an enhanced charge marker, the enhanced charge marker operable to amplify a steric change, an electrostatic change, or a mechanical change, or combinations thereof, during a recognition event of the substance;
exposing the marked substance to a molecule to result in a recognition event of the marked substance with the molecule, the enhanced charge marker amplifying the steric change, electrostatic change, or mechanical change, or combinations thereof, during the recognition event of the marked substance with the molecule; and
non-optically sensing the steric change, electrostatic change, or mechanical change, or combinations thereof of the recognition event of the marked substance with the molecule.

2. The method of claim 1, wherein
the substance marked with an enhanced charge marker is an analyte;
the molecule to which the marked substance is exposed to result in the recognition event of the marked substance with the molecule is a probe; and
the analyte is capable of being differentiated from other substances in a sample if the recognition event occurs between the probe and the marked analyte.

3. The method of claim 1, wherein
the substance marked with an enhanced charge marker is a secondary probe;
the molecule to which the marked substance is exposed to result in the recognition event of the marked substance with the molecule is an analyte; and
the analyte is capable of being differentiated from other substances in a sample if the recognition event occurs between the marked secondary probe and the analyte.

4. The method of claim 3, wherein the analyte is immobilized on a substrate comprising a primary probe, wherein the primary probe is capable of undergoing a recognition event with the analyte.

5. The method of claim 1, further comprising communicating the non-optically sensing of the steric change, electrostatic change, or mechanical change, or combinations thereof, of the recognition event of the marked substance with the molecule to a computing unit.

6. The method of claim 1 wherein marking further comprises selecting an enhanced charge marker comprising an effective charge in the range of −10 to −2 or 2 to 10 if the substance to be marked comprises a net charge in the range of −20 to 20 in a working solution comprising a pH in the range of 3-10.

7. The method of claim 1 wherein marking further comprises selecting an enhanced charge marker comprising an effective charge in the range of −15 to −2 or 2 to 15 if the substance to be marked comprises a net charge in the range of −40 to 40 in a working solution comprising a pH in the range of 3-10.

8. The method of claim 1 wherein marking further comprises selecting an enhanced charge marker comprising an effective charge in the range of −15 to −2 or 2 to 15 if the substance to be marked comprises a net charge in the range of −3 to 3 in a working solution comprising a pH in the range of 6-8.

9. The method of claim 1 wherein marking further comprises selecting an enhanced charge marker comprising an effective charge in the range of −25 to −2 or 2 to 25 if the substance to be marked comprises a net charge in the range of −10 to 10 in a working solution comprising a pH in the range of 6-8.

10. The method of claim 3, wherein the secondary probe is a monoclonal antibody and the enhanced charge marker is peptide nucleic acid (PNA) coupled to the monoclonal antibody via carbodiimide.

11. The method of claim 10, further comprising detecting Prostate Specific Antigen, wherein the monoclonal antibody is anti-Prostate Specific Antigen (anti-PSA).

12. A system, comprising:
a substrate;
at least one primary probe disposed on the substrate that is operable to undergo a recognition event with an analyte; and
a sensor coupled to the substrate, the sensor operable to non-optically detect a steric change, an electrostatic change, or a mechanical change, or combinations thereof, occurring in response to the recognition event of the primary probe with the analyte,
wherein the analyte or a secondary probe, or combinations thereof, is coupled to an enhanced charge marker that is capable of amplifying the non-optical detection by the sensor of the recognition event of the at least one primary probe with the analyte.

13. The system of claim 12, wherein the at least one primary probe or the secondary probe, or combinations thereof, comprise: antibodies, antibody fragments, single-chain antibodies, genetically engineered antibodies, peptide nucleic acids, proteins, peptides, binding proteins, receptor proteins, transport proteins, lectins, substrates, inhibitors, activators, ligands, hormones, neurotranamitters, growth factors, cytokines, carbohydrates, aptamers, lipids, lipid bilayers or charged polymers, or combinations thereof.

14. The system of claim 12, wherein the analyte comprises a(n): acid, base, organic compound, inorganic chemical, amino acid, peptide, polypeptide, protein, glycoprotein, lipoprotein, antibody, sugar, carbohydrate, oligosaccharide, polysaccharide, fatty acid, lipid, hormone, metabolite, growth factor, cytokine, chemokine, receptor, neurotransmitter, antigen, allergen, antibody, substrate, metabolite, cofactor, inhibitor, drug, pharmaceutical, nutrient, biohazardous agent, infectious agent, prion, vitamin, heterocyclic aromatic compound, carcinogen, mutagen, waste product, virus, bacterium, Salmonella, Streptococcus, Legionella, E. coli, Giardia, Cryptosporidium, Rickettsia, spore, mold, yeast, algae, amoebae, dinoflagellate, unicellular organism, pathogen, prion or a cell, or combinations thereof.

15. The system of claim 12, further comprising a package capable of containing a sample comprising the analyte in an inside portion of the package, and wherein the sensor is exposed to the inside portion of the package.

16. The system of claim 12, wherein the sensor comprises a plurality of sensors disposed on the substrate, wherein one or more of the plurality of sensors comprise one or more primary probes extending from the substrate.

17. The system of claim 12, wherein the enhanced charge marker comprises phosphate, carboxylate, amine or sulfonate groups, or combinations thereof.

18. The system of claim 12, wherein the enhanced charge marker comprises a polymer backbone comprising functional groups, where the functional groups comprise; negative DNA (deoxyribonucleic acid) oligomers, negative peptide oligomers, positive peptide oligomers or small molecular weight synthetic polymers, or combinations thereof.

19. The system of claim 18, wherein the small molecular weight synthetic polymers of the enhanced charge marker further comprises positive or negative side groups, or combinations thereof.

20. The system of claim 19, wherein the positive or negative side groups comprise sulfonated aliphatic amines, carboxylated aliphatic amines, aromatic amines aor heterocyclic-organo-metallic complexes, or combinations thereof.

21. The system of claim 12, wherein the sensor comprises a field effect transistor, piezo-electric material, crystal material, ion-sensitive field effect transistor (ISFET), electrolyte-insulator-semiconductor (EIS), amperometric or potentiometer electrode sensor, capacitance sensor, or combinations thereof.
